(19) United States
(12) Patent Application Publication
(10) Pub. No.: US 2014/0239079 A1
(43) Pub. Date: Aug. 28, 2014

(54) HANGING SCENTED BEAD AIR FRESHENER

(71) Applicant: Thomas H. Wolf, Highland Park, IL (US)

(72) Inventor: Thomas H. Wolf, Highland Park, IL (US)

(21) Appl. No.: 14/047,103

(22) Filed: Oct. 7, 2013

Related U.S. Application Data
(63) Continuation-in-part of application No. 13/780,062, filed on Feb. 28, 2013.

(51) Int. Cl. A61L 9/12

(52) U.S. Cl. CPC A61L 9/12
USPC 239/6; 239/58

(57) ABSTRACT

This concerns a hangable air freshener that houses aroma-emanating material, such as a plurality of scented beads. The hangable air freshener has a top wall bearing a hook for suspended placement. When at the opened condition, access is provided to a seal which when compromised or removed allows for passage of fragrance from the scented material, through aroma-permeable area, and out of the air freshener and into the surroundings. The hangable air freshener is manufactured in multiple parts, including an upper part with the hook and a lower part within which components for forming the scented beads are positioned, the parts are sealed together with a seam, and expanding volume beads fill the lower part and move into the upper part.

### CROSS REFERENCE TO RELATED APPLICATION

This is a continuation-in-part of application Ser. No. 13/780,062, filed Feb. 28, 2013, incorporated hereinto by reference.

### DESCRIPTION

1. Technical Field

The present subject matter relates to air fresheners with a source of fragrance in the form of beads or other non-liquid and non-rigid fragrance sources. More particularly, the present subject matter relates to hanging scented air fresheners including a volume of fragrance sources such as aroma-emanating beads and a hook from which the air freshener can be suspended for various household-type uses.

2 Background

Air fresheners are a common source of providing pleasant fragrances at locations in need of freshening. Air fresheners typically are manufactured, shipped, stored in warehouses, displayed at retail, purchased and stored at households or the like before the ultimate user decides to place the air freshener into service. Many air fresheners are configured to rest upon a surface at the desired air freshening location. The present disclosure provides the observation that it would be advantageous to provide air fresheners that remain securely sealed to avoid premature fragrance escape and that do not require a horizontal surface as a resting place once ready for air freshening use.

### SUMMARY

There are several aspects of the present subject matter that may be embodied separately or together in the devices described and claimed below. These aspects may be employed alone or in combination with other aspects of the subject matter described herein, and the description of these aspects together is not intended to preclude these aspects separately or the claiming of such aspects separately or in different combinations as may be set forth in the claims appended hereto.

In one aspect, an air freshener has a contained volume within which non-liquid fragrance sources such as scented aroma emanating beads are positioned and that has a hook by which a user places the device so as to be suspended from a location suitable for hanging.

According to another aspect, a hangable air freshener of fragrance sources such as aroma-emanating scented beads, has an aroma-permeable area fully overlaid by a breakable seal, the seal being removable to expose the aroma-permeable area, the freshener having a hook from which the device can be suspended at a location at which air freshening is desired.

In yet another aspect, an air freshener device containing non-liquid fragrance sources such as scented beads has a top-of-body surface that is oriented at a non-perpendicular angle with respect to sidewalls of the container such that the top-of-body surface is not horizontal when in use and has a hook component at a location elevated with respect to other portions of the container.

In a further aspect, an air freshener contains a volume of scented beads within a container with a transparent and/or translucent wall through which the scented beads are visible and a hook projecting from an upper wall non-parallel to the container bottom wall, whereby the upper wall and a hook projecting thereabove provide a means for suspending the air freshener without having the air freshener rest upon a horizontal surface.

In a further aspect, the device has a two-piece body containing a plurality of scented beads or the like, the device having both pieces of the body secured together at a seam, and a removable or openable seal that allows access to aroma-permeable areas to allow for escape of the fragrance from the scented beads, while a hook of the upper body piece suspends the device without having same resting on a horizontal surface.

In yet a further aspect, a hangable air freshener is provided with a container having sidewalls, a bottom wall, a top wall and an enclosed volume generally therewithin containing a plurality of aroma-emanating beads or the like, the top wall having an aroma-permeable area. A seal panel of a sheet or film has a first condition overlying and sealing the area and a second condition at which the aroma-permeable area is unsealed, which condition allows aroma from the aroma-emanating beads or the like to pass out of the container and into the environment within which the air freshener is positioned. The aroma-permeable area sealed condition of the seal panel can be facilitated by a removable label, a removable plug, a removable sheet, or a combination thereof. The top wall further includes a hook projecting away from the top wall and that is sized and shaped to impart hanging capability to the air freshener.

In another aspect, a hangable air freshener is provided with a container having sidewalls, a bottom wall, a top wall and an enclosed volume generally therewithin containing a plurality of aroma-emanating beads or the like, the container having an upper piece and a lower piece assembled at a seam. Prior to assembly, the lower piece has an access opening through which bead seeds and aroma-emanating liquid enter. Thereafter, the upper and lower pieces are assembled together, the seeds absorb the water to form the aroma-emanating beads, while expanding into the upper piece and generally filling the container. A seal panel of a sheet and/or film plug has a first condition overlying and sealing an aroma-permeable area and a second condition at which the aroma-permeable area is unsealed when the aroma from the aroma-emanating members is allowed to pass out of the container and into the environment within which the air freshener is positioned. The upper piece further includes a hook projecting away from the top wall and being sized and shaped to impart hanging capability to the air freshener. The top wall can be non-parallel to the bottom wall of the container, typically being generally planar and oriented at an acute angle with respect to the bottom wall.

In yet a further aspect, a hangable air freshener is provided with a container having sidewalls, a bottom wall, a top wall and an enclosed volume generally therewithin containing a plurality of aroma-emanating beads or the like, the container having an upper piece and a lower piece assembled at a seam. Prior to assembly, the lower piece has an access opening through which bead seeds and aroma-emanating liquid enter. Thereafter, the upper and lower pieces are assembled together, the seeds absorb the water to form the aroma-emanating beads, while expanding into the upper piece. A seal panel of a sheet or film has a first condition overlying and sealing an aroma-permeable area and a second condition at which the aroma-permeable area is unsealed when the aroma from the aroma-emanating members is allowed to pass out of the container and into the environment within which the air freshener is positioned. The upper piece further includes a hook projecting away from the top wall and being sized and shaped to impart hanging capability to the air freshener. The top wall can be non-parallel to the bottom wall of the container, typically being generally planar and oriented at an acute angle with respect to the bottom wall. The hook is unitary with the cover and positioned at a location of the top wall closer to one end than to the opposing end of the top wall.

In another aspect, a hangable air freshener is provided with a container having sidewalls, a bottom wall, a top area and an enclosed volume generally therewithin, the top area having a first end portion and a second end portion, a plurality of aroma-emanating beads being within the enclosed volume. The top area has an aroma-permeable area and a seal panel sheet plug having a first condition overlying and sealing the aroma-permeable area, the seal sheet plug having a second condition at which the aroma-permeable area is unsealed, and aroma from the aroma-emanating beads is allowed to pass out of apertures, which can be one or more openings or aroma-permeable membranes at the top area of the container and into the environment within which the air freshener is positioned. The seal panel sheet plug has at least one protrusion closing one or more of the top area apertures, same being instrumental in the overlying and sealing first condition. A hook component of the cover projects away from the top area of the container, the hook component being sized and shaped to impart hanging capability to the air freshener.

In another aspect, a hangable air freshener is provided with a container having sidewalls, a bottom wall, a top area and an enclosed volume generally therewithin, the top area having a first end portion and a second end portion, a plurality of aroma-emanating beads being within the enclosed volume. The top area has an aroma-permeable area and a seal panel sheet plug having a first condition overlying and sealing the aroma-permeable area, the seal panel sheet plug having a second condition at which the aroma-permeable area is unsealed, and aroma from the aroma-emanating beads is allowed to pass out of one or more apertures at the top area of the container and into the environment within which the air freshener is positioned. The seal panel has at least one projection closing one or more of the top area apertures, same being instrumental in the overlying and sealing first condition. A hook component of the cover projects away from the top area of the container, the hook component being sized and shaped to impart hanging capability to the air freshener. The container sidewalls include opposing sidewalls, one sidewall being at an acute angle with the first end portion of the top area, its opposing sidewall being at an obtuse angle with the second end portion of the top area, and the hook component is at a location of the cover closer to said first end portion than to said second end portion of the top area.

In another aspect, a hangable air freshener is provided with a container having sidewalls, a bottom wall, a top area and an enclosed volume generally therewithin, the top area having a first end portion and a second end portion, a plurality of aroma-emanating beads being within the enclosed volume. The top area has an aroma-permeable area and a seal panel or sheet plug having a first condition overlying and sealing the aroma-permeable area, the seal panel or sheet plug having a second condition at which the aroma-permeable area is unsealed, and aroma from the aroma-emanating beads is allowed to pass out of one or more apertures at the top area of the container and into the environment within which the air freshener is positioned. The seal panel has at least one protuberance or projection closing one or more of the top area apertures, same being instrumental in the overlying and sealing first condition. A hook component of the cover projects away from the top area of the container, the hook component being sized and shaped to impart hanging capability to the air freshener. The container includes an upper part and a lower part which are joined together by a seam assembly typically including a seal arrangement that can exhibit snapping action, after positioning aroma materials into the container. In select embodiments, seam disassembly allows separation of the upper and lower parts at the seam for removing spent beads, re-filling and re-closing such seam.

In another aspect, a hangable air freshener is provided with a container having sidewalls, a bottom wall, a top area and an enclosed volume generally therewithin, the top area having a first end portion and a second end portion, a plurality of aroma-emanating beads being within the enclosed volume. The top area has an inset aroma-permeable area and a seal panel or sheet plug having a first condition overlying and sealing the aroma-permeable area, the seal panel or sheet plug having a second condition at which the aroma-permeable area is unsealed, and aroma from the aroma-emanating beads is allowed to pass out of one or more apertures at the top area of the container and into the environment within which the air freshener is positioned. The seal panel has at least one protuberance closing one or more of the top area apertures, same being instrumental in the overlying and sealing first condition. A hook component of the cover projects away from the top area of the container, the hook component being sized and shaped to impart hanging capability to the air freshener. The seal sheet plug includes a digital grasping component by which the seal panel or sheet plug can be lifted off the one or more openings by application of digital forces.

### BRIEF DESCRIPTION OF THE DRAWINGS

FIG. 1 is a perspective view of a hangable air freshener in accordance with one embodiment, including scented beads therewithin;

FIG. 2 is a side elevational view of the embodiment of FIG. 1, showing a sidewall wider than its adjoining sidewalls;

FIG. 3 is a side elevational view of the embodiment of FIG. 1, showing one of the opposing narrower sidewalls and with sidewall labeling shown in phantom as an illustrative configuration;

FIG. 4 is a longitudinal view, partially elevational and partially in exploded cross-section through the embodiment of FIG. 3;

FIG. 4A is an enlarged elevational and cross-sectional view through a part of the upper portion of FIG. 4;

FIG. 5 is a top plan view of FIG. 1;

FIG. 6 is a bottom plan view of FIG. 1;

FIG. 7 is an exploded perspective view of a second embodiment showing the seal exploded from the top wall;

FIG. 8 is a partial longitudinal cross-sectional view through an upper portion of FIG. 7;

FIG. 9 is a partial transverse cross-sectional view through the upper portion of FIG. 7;

FIG. 10 is an exploded partial perspective view of a third embodiment incorporating a pull-tab removable plug;

FIG. 11 is an exploded partial perspective view of a fourth embodiment incorporating a pull-tab removable plug;

FIG. 12 is an exploded perspective view of a fifth embodiment showing a seal exploded from the top wall;

FIG. 13 is a plan view of the top panel with aroma-permeable areas similar to those of FIG. 12;

FIG. 14 is a plan view of the top panel with another embodiment of aroma-permeable areas; and

FIG. 15 is an exploded perspective view of an embodiment prior to assembly and illustrates a method stage prior to closure of the seam and assembly and prior to expansion of seeds by aroma-supplying liquid into the filled container as in other figures.

### DETAILED DESCRIPTION OF THE ILLUSTRATED EMBODIMENTS

The embodiments disclosed herein are exemplary only, and the subject matter described herein may be embodied in various forms. Therefore, specific details disclosed herein are not to be interpreted as limiting the scope of the subject matter as defined in the accompanying claims.

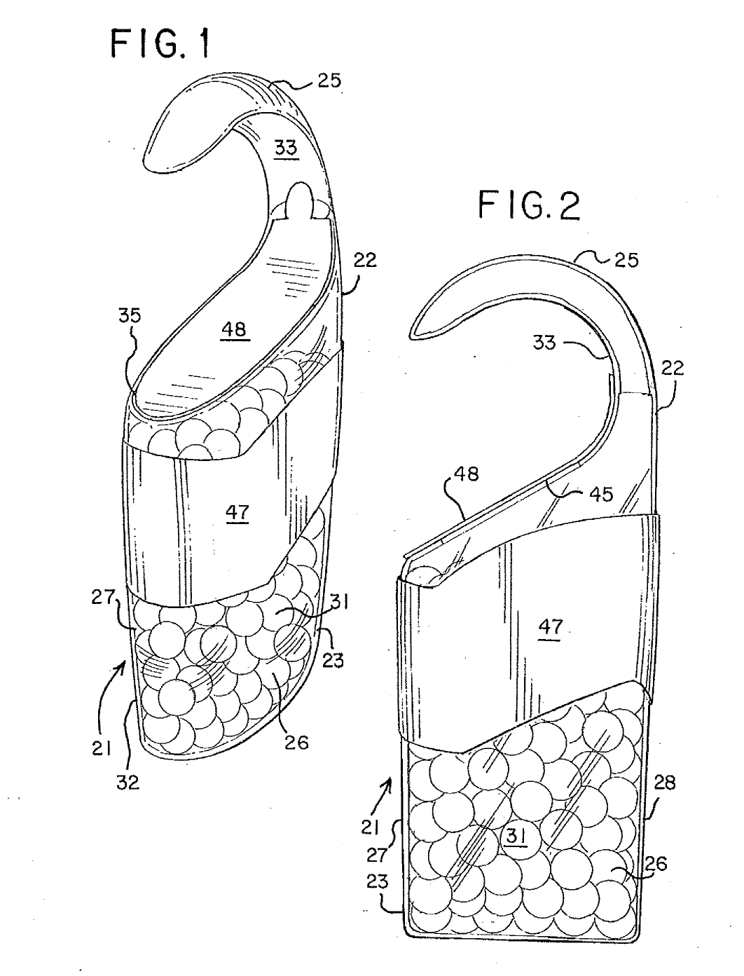

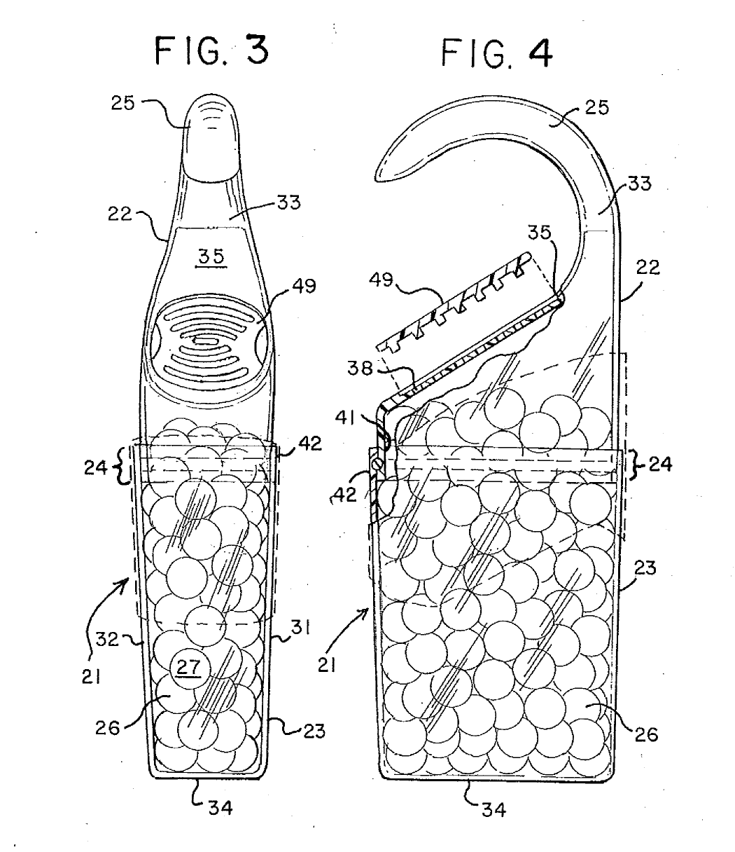

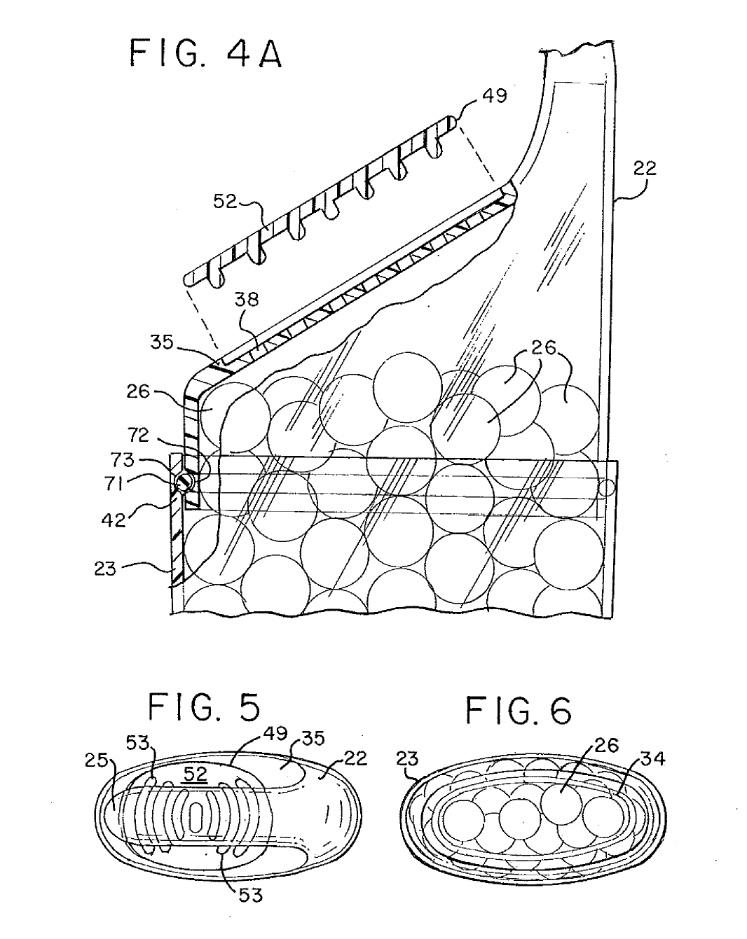

An embodiment of a hangable air freshener is depicted generally as 21 in FIG. 1, FIG. 2, FIG. 3 and FIG. 4. This includes an upper body piece 22 and a lower body piece 23, joined at a seam or seam assembly generally designated at 24 (FIG. 3 and FIG. 4). The upper body piece includes a hook component 25 projecting upwardly (as seen in the drawings) from the upper body piece 22.

A plurality of non-liquid fragrance sources 26 are positioned within and fill the lower body piece 23 of the container while typically substantially filling the upper body piece 22 of the container. Typically this substantial filling of the upper body piece will have non-liquid fragrance sources 26 at least partially in a hollow hook portion of the upper body piece 22, for example, its stem.

These non-liquid fragrance sources can be scented beads. When used herein, the term “scented beads” is used at various times to encompass non-liquid fragrance sources whether or not spherical in shape. Scented beads 26 are of generally known construction, composition and properties, being impregnated with fragrance material that releases from the bead when the bead is exposed to air or the environment. These scented beads are known to be available in a variety of aromas and colors. Various specific properties of these scented beads are known to one of ordinary skill in the art. They may or may not be substantially spherical.

In some embodiments, it will be observed that at least one wall of the container is non-opaque, such as being translucent or transparent, whereby the presence of scented beads or other non-liquid fragrance sources 26 within the container can be visible and understood to be present therewithin. In one embodiment, all of the sidewalls can be transparent, as generally shown in FIG. 1 for example. The sidewalls include opposing narrower sidewalls 27 and 28, and opposing wider sidewalls 31 and 32. Any one of sidewalls 27, 28, 31, 32 or a plurality of them, or a portion of them, can be non-opaque depending upon the extent that one wishes to have the scented beads generally visible within the container 22. Also possibly non-opaque can be the top portion or stem 33 and/or the bottom wall 34. In an embodiment, the upper body piece 22 is opaque and the lower body piece 23 is non-opaque, or vice-versa. Any of these non-opaque components may be transparent, translucent or other condition that permits features of the scented beads to be discernible from outside of the closed container.

The hook component is illustrated in the drawings as having an overall structure typical of a hook for hanging a device within a household context. As illustrated, the hook component 25 that is shown in the drawings receives the support from which it is to be hung at a location above the upper body piece 22 of the container, the curve of the hook component being over the container as opposed to any direction oriented away from the container. The particular shape of the hook component is variable; for example, they can be generally curved, partially curved as shown in FIG. 8, and/or have walls or surfaces that are generally flat as in FIG. 1 and meet at angles with other walls or surfaces. Generally, the hook component will have a curved configuration where the inside surface of the hook component tends to be more compatible with a typical household member from which the hangable air freshener can be hung, such as a dowel or rod within a closet or the like.

The hangable air freshener 21 can include an inclined top wall 35. In the illustrated embodiments, this inclined top wall 35 is not parallel to bottom wall 34, being at an obtuse angle with respect to narrow sidewall 27. In some embodiments, the top wall 35 is at an obtuse angle with respect to top portion or stem 33 of hook component 25. When such an arrangement is provided, the top wall 35 meets one of the opposing narrower sidewalls 28 at an acute angle and meets the other opposing sidewall 27 at an obtuse angle (both angles being measured from the inside of the container), thereby creating the slanted or angled top portion of the container that is illustrated. In the embodiment illustrated in FIG. 2, it will be appreciated that the top wall 35 is non-parallel with respect to the bottom wall 34. Stated otherwise, the top wall is spaced farther from the bottom wall for a distance that is longer along opposing narrow sidewall 28 than along opposing narrow sidewall 27. When desired, the top wall 35 of the container can be considered an extension of the opposing wider sidewalls 31, 32 in a generally curved fashion. There is no requirement that the top wall be itself substantially flat. It merely is to be of a shape that will accommodate an embodiment of the seal and aroma-permeable area.

In the illustrated embodiments, components that are included in formation of seam 24 are found at a bottom skirt or rim 41 of the upper body piece 22 and at a top rim or skirt 42 of the lower body piece 23. Before upper body piece 22 is sealed onto the lower body piece 23 to form the seam or seam assembly 24, the lower body piece rim 42 provides an access opening 36 (FIG. 15). This access opening can be used to fill the container with the scented beads 26 or precursors thereto either at the factory or by the consumer when a refillable hangable air freshener is desired. In the illustrated embodiment, a method is followed that is illustrated in FIG. 15 whereby polymer seeds 43 that expand when exposed to liquid are positioned within the lower body piece 23, passing through access opening 36. Also added in a similar manner is a volume of aroma-importing liquid 44 such as scented water that expands and adds aroma to the seeds 43 to form the beads 26.

Thereafter, in this method, seam or seam assembly 34 is formed. The seam typically has the upper body piece skirt 41 enter the lower body piece rim 42. Seam formation can include sealing, force-fitting and/or “snapping” together of the upper and lower body portions at the upper body piece bottom skirt 41 and the lower body piece top rim 42. The aroma-imparting liquid 44 is absorbed by the seeds 43, which then expand into the scented beads 26, substantially filling the lower and upper body pieces 23, 22. Partial upper body piece filling can be practiced. Typically this action is facilitated by gentle shaking of the sealed container. An example of the extent of filling is seen in the various figures.

Top wall 34 includes one or more aroma-permeable areas 45. Prior to initial use by the consumer, all such areas are sealed so that the fragrance of the scented beads 26 will remain within the container during warehousing, transporting, retailing and storage in the household before use. A suitable manner of achieving this objective is to include a seal panel, sheet or film 37, as in FIG. 8, FIG. 9 and FIG. 12. Seal panel 37 can take the form of a thin film that provides an airtight seal such as found in over-the-counter pharmaceutical compositions, certain air freshener packaging, food products and the like. These can be of a so-called foil type and/or be of multiple layers or laminates having barrier capabilities in one or more layers. Barrier layers can be metallic, polymeric or combinations thereof.

The seal panel 37 of this embodiment is affixed to surfacing along the top wall, for example around the aroma-permeable areas, by adhesive means, heat sealing, sonic welding, or other manner of attachment known in the art. When thus attached, the seal sheet 37 provides barrier functions as noted herein, and when needed secures the scented beads and their aroma-imparting liquid within the compartment. In addition, the seal sheet can be frangible so that the seal can be compromised and/or removed partially or completely in order to reduce or remove barriers to fragrance dissemination out of the container confining the scented beads. It is generally preferred that this frangibility feature be such that the seal panel 37 is removable, penetrable or otherwise compromisable without requiring specially designed tools, usually requiring something of only moderate sharpness such as a pointed object or a fingernail, or by concerted digital force imparted to the seal sheet by the user.

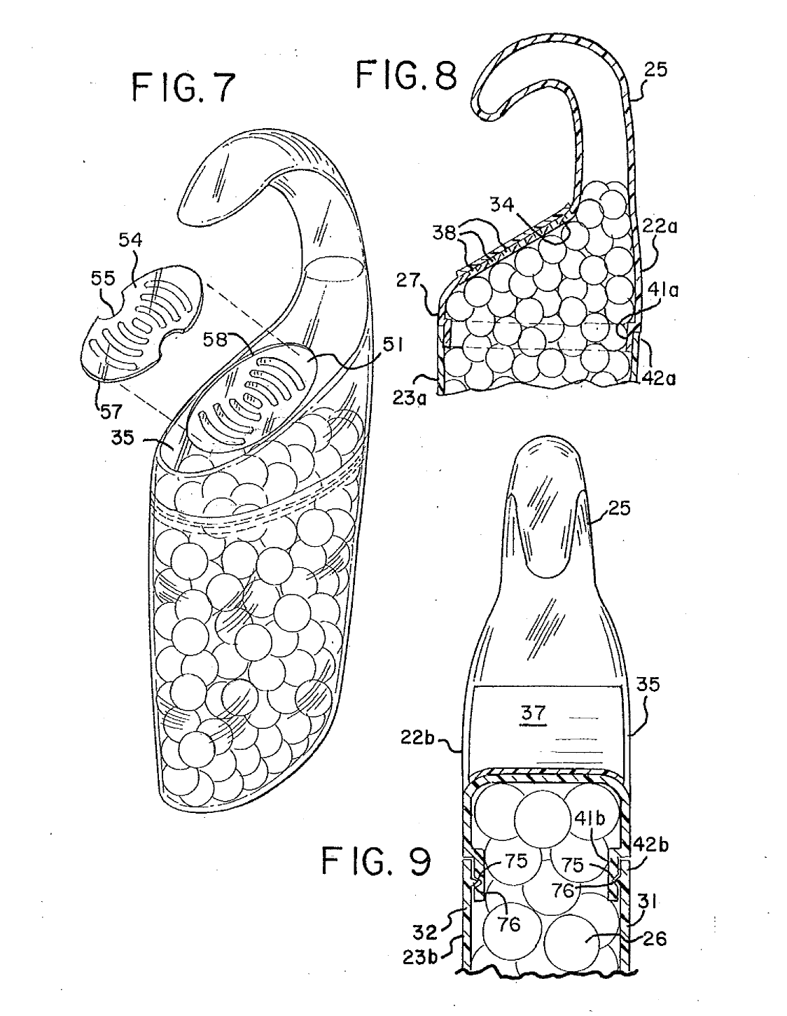

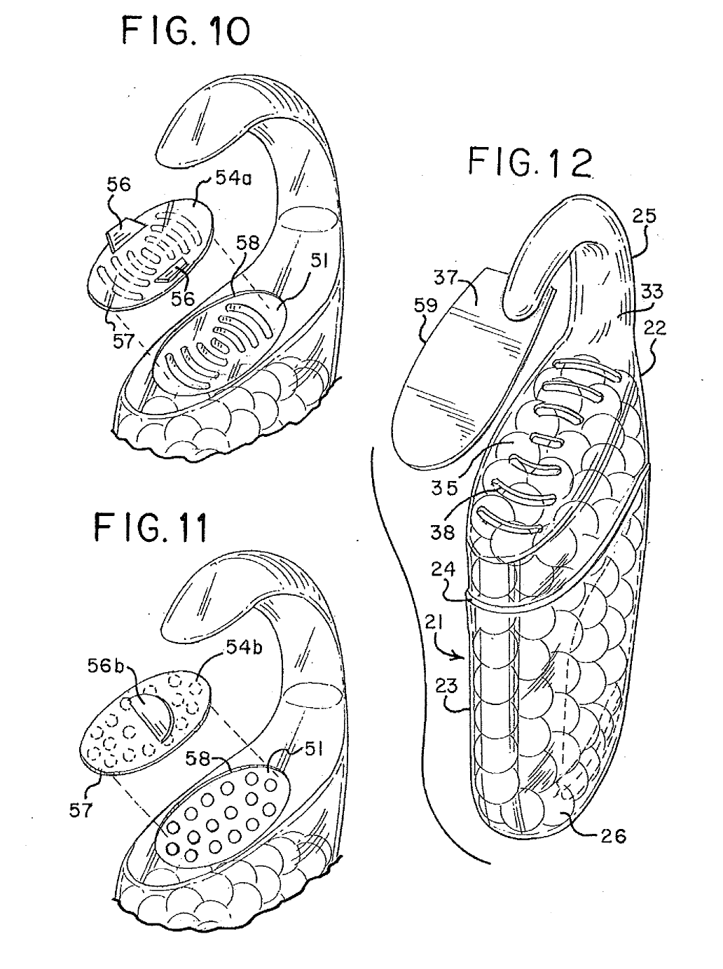

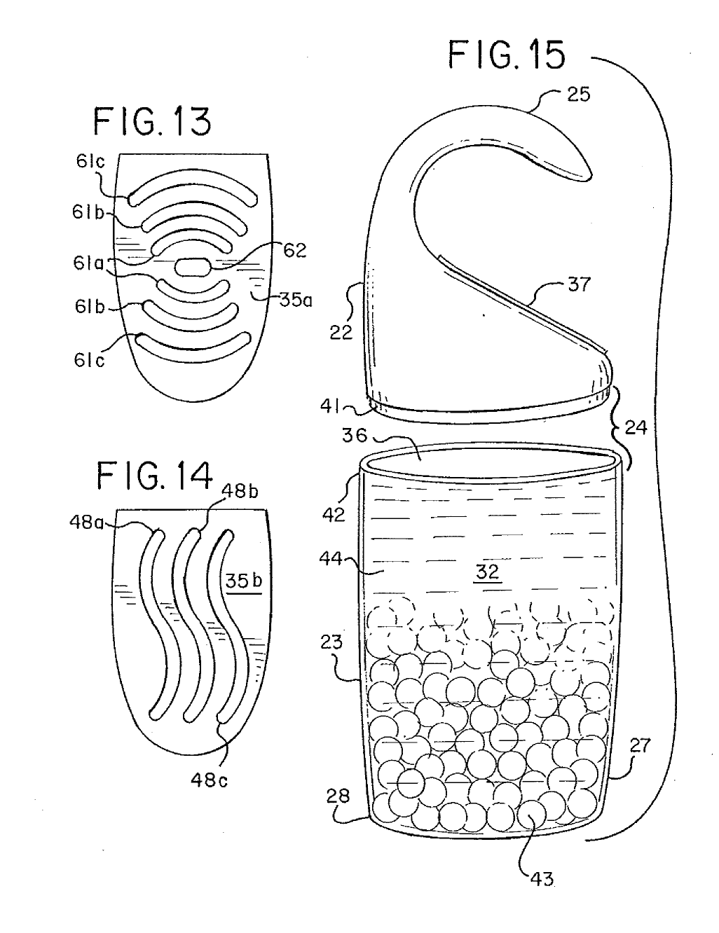

With more particular reference to the aroma-permeable areas, same may include one or more apertures 38 (e.g., FIG. 4 and FIG. 4A) completely through the top wall. FIG. 7, FIG. 10, FIG. 12, FIG. 13 and FIG. 14 show different respective patterns of multiple aroma-permeable areas of the top wall 35. These areas are apertures completely through top wall 35 or a thinned-down portion of the top wall or an aroma-permeable membrane. For example, the various patterns of areas can be thinned-down areas or otherwise aroma-transmitting areas or membranes that are adequate to release aroma from the beads 26 into the surroundings of the hangable air freshener 21.

Particular reference is made to the first embodiment that is shown in FIG. 1, FIG. 2, FIG. 3, FIG. 4, FIG. 4A, FIG. 5 and FIG. 6. Included is hanging air freshener body label 47 illustrating a particular label configuration, as well as aroma access label 48 that covers an aroma-permeable area that is a membrane 45 (FIG. 2) between the label 48 and the interior containing the beads 26. Alternatively, the aroma-permeable area is a combination of a plurality of apertures 38 and a plug 49 (FIG. 3, FIG. 4, FIG. 4A). As perhaps best seen in FIG. 4A, the top wall 34 features a thinned-down location or recess 51. The plug 49 has a backbone panel or sheet 52 and one or more protuberances 53 thereof. The recess 51 accommodates the backbone panel or sheet 52 while the protuberances 53 enter and close the apertures 38. For drawing clarity, the recess 51 has a depth less than the thickness of the backbone panel 52; however, it will be appreciated that providing this depth and thickness with a substantially equal respective dimensional values will allow the plug to be substantially flush with respect to the outer face of the top wall 35.

Whether the plug 49 is fully flush within the recess 51 or not, the aroma access label 48 (FIG. 1) is adhesively or otherwise secured to the top wall 35 in a manner to filly enclose the plug 49. The aroma access label 48 provides tamper evidence functions and freshness functions as an added barrier between the aroma beads 26 and the environment outside of the hangable air freshener 21. Aroma access label 48 also provides the functions of product identification, marketing and appearance enhancement. These latter functions also are provided by the body label 47, an example of the shape of same being shown as an opaque label in FIG. 1 and FIG. 2. Alternatively, the body label can be transparent and/or translucent in whole or in part, allowing visualization of the scented beads at such locations.

Various embodiments specifically shown herein include one or more apertures 38 partially or completely through the top wall 35 of the upper body piece 23. For purposes of illustration, multiple aperture patterns are illustrated in the drawings. Also, the aroma-disseminating ability of the apertures 38 are blocked until use of the device is intended. This blocking can be accomplished by the aroma access label 48 or by the combination of the plug 49 and aroma access label 48, or they can be blocked by a removable seal panel, sheet or film 37 such as shown in FIG. 8, FIG. 9 and FIG. 12. It is also possible to provide a seal panel, sheet or film that fits within recess 51 that does not have any protruberances, or same can be provided as seal panel or film plug 54 as shown in FIG. 7. This seal panel or film plug 54 includes one or more edge indents 55 to facilitate removal from the tap wall 35, for example, from out of the recess 51, as shown in FIG. 7. This particular seal panel or film plug 54 is substantially transparent, and the aperture patterns are also present on or in the plug 54.

A non-transparent seal panel or film plug 54a is illustrated in FIG. 10. This arrangement adds a plurality of tabs 56 to facilitate digital removal of the plug 54a when blocking of the aroma-permeable areas is no longer intended. FIG. 11 illustrates a further example of a seal panel or film plug 54b, showing a single tab 56b provided to facilitate plug removal and unblocking of the aroma-permeable areas. In the embodiments of FIG. 10 and FIG. 11, the respective seal panel or film plug is securely positioned within recess 51.

The seal panel or film plug, whether with or without protruberances, typically is sized and shaped so as to snuggly fit within recess 51 (when provided), thereby enhancing the security and aroma transmission blocking capabilities of the illustrated systems. In these instances, all or most of the perimeter of the seal panel or film plug is shaped and dimensioned so as to be substantially the same as the perimeter of the recess. Depending upon the compressibility and/or flexibility of the polymeric material or materials of the plug and of the recess perimeter walls, the perimeter dimension of the plug can be slightly less than the perimeter dimension of the recess 51. An objective is to provide a removable force fit between the respective perimeter walls of the plug and recess. In the illustrated embodiments, the plug perimeter wall 57 and the recess perimeter wall 58 are generally oval in shape, and it will be appreciated that other shapes are possible.

Continuing with the discussion of the aroma permeable areas, different shapes are contemplated. In those instances where a plug with protuberances is part of the aroma blocking function, maximum blocking is achieved when the protuberances are shaped and sized to be substantially the same as those of the apertures, especially where force-fit conditions are exhibited between the protuberances and the apertures. One illustrated shape pattern is shown in FIG. 3, FIG. 5 and FIG. 7. This pattern is specifically depicted in FIG. 13, shown on a top wall 35a of the embodiment of FIG. 12. Although not explicitly shown in FIG. 12, the seal panel or film 37 of this embodiment can have a complementarily shaped recess on the top wall 35a, in which event the perimeter 59 of seal sheet or film will substantially match same of the indent (not shown in FIG. 12) in a manner discussed elsewhere herein.

Referring more specifically to the aperture patterns illustrated, FIG. 13 depicts a plurality of generally arcuate aroma-permeable area pairs 61a, 61b, 61c of varying sizes. The illustrated pattern shows the arcuate aroma-permeable pairs 61a, 61b, 61c with an increasing-size pattern from center to edge. This increasing-size pattern increases the area of the pairs, which increase can be an increase in length and/or in width. A central aroma-permeable area 62 also is shown. FIG. 14 illustrates another variation of multiple aroma-permeable areas of a top wall 35b. FIG. 14 depicts a plurality of generally S-shaped aroma-permeable areas 63a, 63b, 63c that are of substantially the same shape and size uniformly spaced apart.

Various approaches of the seam assembly are illustrated for securely joining the upper body piece 22 and lower body piece 23 in forming the hangable air freshener 21. With the embodiment illustrated in FIG. 3, FIG. 4 and FIG. 4A, a resilient member 71, such as an O-ring, is positioned between bottom skirt 41 and top rim 42 in order to more securely join lower body piece 23 and upper body piece 22. It will be appreciated that the resilient sealing member 71 can seat within a groove or grooves within either or both of the bottom or top skirts or rims. FIG. 4A shows a groove in both. More particularly, a groove 72 is illustrated in bottom skirt 41 of the upper body piece 22, while a groove 73 is shown in the top rim 42 of lower body piece 23.

FIG. 8 shows a bottom skirt or rim 41a of the upper body piece that is inset with respect to the rest of the upper body piece 22a, while the top rim or skirt 42a of the lower body piece 23a is of substantially the same size so as to be substantially flush with each other. Although not specifically illustrated, a reversed relationship is possible, wherein the top rim of the lower body piece is inset and the bottom skirt of the upper body piece is straight-walled. In another variation, the top rim of the lower body piece is larger in perimeter than the rest of the lower body piece, thereby providing a ledge (not shown) onto which the bottom skirt of an upper body piece rests.

In a variation illustrated in FIG. 9, interference elements are added to the skirt and rim interface such as that of FIG. 8 or of the alternative skirt and rim configurations discussed herein. In the specific arrangement illustrated in FIG. 9, the interference elements take the form of an indent receiving a complementary detent. A specifically illustrated arrangement provides an indent 75 on the inside surface of the skirt 41b of the upper body piece 22b and a detent 76 in the rim 42b of the lower body piece 23b. These interference elements help to provide a snapping action to indicate when the upper and lower body pieces are properly seated with respect to each other. The interference elements also maintain the assembly of the upper and lower body pieces and provide a supplemental barrier to aroma escape at the seam between the upper and lower body pieces.

In use, the user of the hangable air freshener compromises the seal by removing the seal panel such as the plug (and label when provided) or seal sheet (or a majority of the seal sheet), which can be facilitated by a lift assist mechanism such as the use of a peel tab digital recess illustrated herein. Alternatively, the user can fracture the seal panel or film (when provided) or a thinned-down portion of an aperture after label removal by a simple sharp object, by a fingernail, or by the user of digital force in order to create an opening or fracture in the seal or thinned-down portion, which in many instances is adequate to allow sufficient passage of fragrance from the scented beads and into the atmosphere surrounding the hangable air freshener. For example, the seal can be breached only at an area above one or more of the aroma-permeable areas, which is especially facilitated when the aroma-permeable areas are apertures, in which case the breach (such as of the seal sheet 37) is generally into the aperture or apertures. As previously noted, the aperture or apertures can have a thinned-down portion or web of polymer rather than be a fully open aperture. Alternatively, where the web has aroma permeability or a membrane is provided, same can remain unaltered and still permit aroma permeation. Alternatively, the web can be broken to allow or enhance aroma permeation.

By whatever mechanism is used to allow aroma passage, fragrance from the scented beads 26 is able to escape from the container by way of the aperture or apertures, at the discretion of the user. At the same time, the hangable air freshener is in a condition for hanging, such as over a bar or rod of a closet or any other location in need of air freshening as provided by fragrance from a passive source, the illustrated passive source being scented beads 26 and fragrance escape through the thus unsealed aroma permeable areas or aperture or apertures.

With at least some of the illustrated embodiments, the hangable air freshener can be of a refillable variety. More specifically, as specifically illustrated in FIG. 15, after the seal sheet or film has been removed or otherwise substantially compromised for a time after which the fragrance-imparting character to the air freshener has ended, the user can remove spent scented beads out of the container through the access opening 36 once the seam is undone. Thereafter, polymeric seeds 43 and scented liquid 44, or fresh scented beads 26, can then be inserted into the container through the access opening. This is followed by return of the sealed seam such as by re-locating the resilient sealing member and/or by snapping action, such as by reengaging the indent and detent. Thereafter, the hangable air freshener can be repositioned where desired within a household or other location for air freshening purposes.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features thereof, including combinations of features that are individually disclosed or claimed herein.

1. A hangable air freshener, comprising:
a container body having sidewalls, a bottom wall, a top area, and an enclosed volume generally therewithin;
a plurality of aroma-emanating beads within the enclosed volume;
an area of at least one said wall of the container is non-opaque thereby allowing observation of at least a portion of at least some of the aroma-emanating beads with an unaided eye;
said top area has at least one aroma-permeable area to permit passage therethrough of aroma of the beads from the enclosed volume to the environment outside the air freshener;
a seal panel having a first condition overlying and sealing said aroma-permeable area, the seal panel having a second condition at which the aroma-permeating area is unsealed;
said container body comprising an upper body piece and a lower body piece separate from each other that are assembled together at a seam of said container body to provide the enclosed volume; and
a hook component of the container upper body piece that projects away from the top area, the hook component being sized and shaped to impart hanging capability to the air freshener.

2. The hangable air freshener of claim 1, wherein said seam extends the combined periphery of the sidewalls along a plane generally parallel to the bottom wall.

3. The hangable air freshener of claim 2, wherein the seam comprises a skirt at a bottom portion of the upper body piece engaged with a rim of a top portion of the lower body piece.

4. The hangable air freshener of claim 3, wherein the seam includes an indent of one of said body pieces and an extension of the other of said body pieces, the indent and extension being snappingly complementary to each other.

5. The hangable air freshener of claim 4, wherein the indent extends the periphery of one of said body pieces, and said extension is an open peripheral rim of the other of said container pieces.

6. The hangable air freshener of claim 4, wherein the indent extends the periphery of one of said body pieces, and said extension is at least one detent projecting from the other body piece.

7. The hangable air freshener of claim 3, wherein at least one of the skirt or rim has an annular groove, and the seam further includes a resilient sealing member at least partially within said groove.

8. The hangable air freshener of claim 1, wherein said seal panel includes a removable plug with an overlying removable seal sheet, the removable plug fully blocks the aroma-permeable area when the seal panel is at the first condition.

9. The hangable air freshener of claim 8, wherein said aroma-permeable area includes a recessed volume below the surface of the top area, said removable plug has a backbone sheet and at least one protuberance from the backbone sheet, and the protuberance enters into and blocks the recessed volume of the aroma-permeable area.

10. The hangable air freshener of claim 8, wherein said removable plug has digital grasping members selected from the group consisting of a tab, a plurality of tabs, an edge indent, a plurality of edge indents, and combinations thereof.

11. The hangable air freshener of claim 1, wherein said sidewalls include two first opposing sidewalls and two second opposing sidewalls narrower than the first opposing sidewalls, the top area has two ends, a first such end being at an acute angle with one of said second opposing sidewalls, the top area has two ends, a first such end being at an acute angle with one of said second opposing sidewalls, the hook component is at a location of the container body upper portion piece closer to said first end than to said second end of the support surface, and the hook component is at least partially hollow including a hollow stem volume thereof that is a portion of said enclosed volume containing aroma-emanating beads.

12. The hangable air freshener of claim 1, wherein said aroma-permeable area comprises a plurality of complementarily shaped elongated curved areas separate from each other.

13. The hangable air freshener of claim 1, wherein a plurality of the aroma-permeable areas are provided and are openings through the thickness of the top area.

14. The hangable air freshener of claim 1, wherein the aroma-permeable area includes a membrane, and a seal sheet overlies the membrane at the first condition.

15. The hangable air freshener of claim 1, wherein the aroma-emanating beads are formed in situ from expandable polymeric seeds and aroma-infused liquid.

16. The hangable air freshener of claim 1, wherein the seam is openable and re-sealable.

17. A hangable air freshener, comprising:
a container having sidewalls, a bottom wall, a top area and an enclosed volume generally therewithin, the top area having a first end portion and a second end portion;
a plurality of aroma-emanating beads within the enclosed volume;
at least one said wall of the container is non-opaque thereby allowing observation of at least a portion of at least some of the aroma-emanating beads with an unaided eye;
said top area has at least one aroma-permeable area to permit passage therethrough of aroma of the beads from the enclosed volume to the environment outside the air freshener;
a seal panel having a first condition overlying and sealing said aroma-permeable area, the seal panel having a second condition at which the aroma-permeating area is unsealed and said seal panel includes a removable plug with an overlying removable seal sheet, the removable plug fully blocks the aroma-permeable area when the seal panel is at the first condition;
said container body comprising an upper body piece and a lower body piece separate from each other that are assembled together at a seam of said container body to provide the enclosed volume, and said seam extends the combined periphery of the sidewalls along a plane generally parallel to the bottom wall;
a hook component of the container body upper piece body piece that projects away from the top area, the hook component being sized and shaped to impart hanging capability to the air freshener, and the hook component is at least partially hollow including a hollow stem volume thereof that is a portion of said enclosed volume containing aroma-emanating beads;
said sidewalls include opposing sidewalls, one sidewall being at an acute angle with the first end portion of the top area, its opposing sidewall being at an obtuse angle with the second end portion of the top area, and the hook component is at a location of the cover closer to said first end portion than to said second end portion of the top area; and
the seam comprises a skirt of one of said body pieces engaged with a rim of the other of said body pieces, and the rim has an annular groove, and the seam further includes a resilient sealing member at least partially within said groove.

18. The hangable air freshener of claim 17, wherein said aroma-permeable area includes a recessed volume below the surface of the top area, said removable plug has a backbone sheet and at least one protuberance from the backbone sheet, and the protuberance enters into and blocks the recessed volume of the aroma-permeable area.

19. A method of providing a hangable air freshener, comprising:
positioning expandable polymeric seeds into a lower body piece of a container body, inserting aroma-infused liquid into the lower body piece;
positioning an upper body piece of the container body over the lower body piece so as to fully enclose the lower body piece at its upper rim to form an enclosed volume, the upper body piece having a hook component projecting away from the lower body piece, as well as at least one aroma-permeable area to permit passage therethrough of aroma from inside the enclosed volume to the environment outside the air freshener which passage is blocked by a removable seal panel overlying the aroma-permeable area;
assembling the upper and lower portion pieces together to form a seam of the container body to provide an enclosed volume of a hangable air freshener; and
gently manipulating the thus-formed seamed container body until the aroma-emanating beads are formed in situ within the enclosed volume from the polymeric seeds and aroma-infused liquid.

20. The method according to claim 19, further including:
opening the seam and removing spent aroma-emanating beads from the enclosed volume;
inserting into the lower body piece the aroma-emanating material; and
re-forming the seam to reinstate the enclosed volume.
